(19) United States
(12) Patent Application Publication
(10) Pub. No.: US 2009/0035389 A1
(43) Pub. Date: Feb. 5, 2009

(54) TARGETED PROTEIN CAGES

(75) Inventors: Brian K. Campion, Burlingame, CA (US); Marvin I. Siegel, Blue Bell, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: SpeciGen Inc., Burlingame, CA (US)

(21) Appl. No.: 12/035,928

(22) Filed: Feb. 22, 2008

Related U.S. Application Data
(60) Provisional application No. 60/891,457, filed on Feb. 23, 2007.

(51) Int. Cl. A61K 47/42; A61K 31/337; A61K 33/36; A61K 31/7105; A61P 35/00

(52) U.S. Cl. 424/623; 514/773; 514/449; 514/44

(57) ABSTRACT

The present invention provides targeted protein cages for the specific delivery of a variety of agents to cells and tissues and methods of use. The targeted protein cages have exterior targeting moieties and therapeutic or imaging agents which are encapsulated within the protein cages or are located on the exterior surfaces of the protein cages.

### CROSS-REFERENCES TO RELATED APPLICATIONS

The present application claims priority to U.S. Ser. No. 60/891,457, filed Feb. 23, 2007, herein incorporated by reference.

### BACKGROUND OF THE INVENTION

Previous efforts have been directed to the development of protein “shells” that may be loaded with different types of materials. As an example, viral coat proteins have been used to form protein shells that encapsulate non-viral materials; see U.S. Pat. Nos. 6,180,389 and 6,984,386, and U.S. Patent Application Publication 2004/0028694. Moreover, ferritin protein cages have been described that can be loaded with uniform materials. The adaptability of such proteins for this use results from their ability to form self-assembling shells. Additionally, many protein cages thus formed have natural controllable channels through which materials can pass, thus allowing for the reversible or irreversible loading of the protein cages. Among the types of materials that have been loaded into such protein cages are various organic, inorganic, and organo-metallic materials.

Given the potentially enormous range of materials that may be encapsulated in protein cages, such compositions have great potential for a variety of diagnostic and therapeutic applications by functioning as Trojan horses after gaining access to cells and tissues. However, to fully exploit the potential of reagent-loaded protein cages, better ways of targeting these compositions to the specific cells and tissues where a particular agent exerts its effects are needed. In the case of a chemotherapeutic agent, for instance, the ability to target drug-containing protein cages to cancer cells would be expected to lead to increased tumor cell cytotoxicity while avoiding damage to normal cells. In the case of a diagnostic imaging agent, the ability to specifically target such protein cages to particular tissues or organs will result in earlier detection and improved diagnostic capability. The present invention meets these and other needs.

### BRIEF SUMMARY OF THE INVENTION

In an embodiment, the present invention provides a proteinaceous composition comprising a protein cage having a targeting moiety and a payload encapsulated within the protein cage. In some aspects, the targeting moiety can be an antibody or fragment thereof, a ligand for a receptor, a carbohydrate, a lipid, or a polynucleotide. In some aspects, the antibody can be a monoclonal antibody which can be humanized.

In further aspects, the antibody or fragment thereof binds a cell surface molecule, which can generally be a growth factor receptor, hormone receptor, lymphocyte surface marker, cell-specific differentiation marker, or cell adhesion molecule, among others. Specific examples of such cell surface molecules include CTLA4, CD4, CD20, EGFR, CD30, PSMA, CD89, integrin, mannose receptor/hCG#, PD1, PDGFR, CD33, CD 5, HER2, CEA, CD13, CD14, CD15, CD33, CD5, CD21, CD19, CD20, CD2, CD3, CD8, CD16, CD 56, CD22, CD56, and IGFR.

Additionally, the antibody or fragment thereof may bind a tumor antigen expressed on a cancer cell derived by mutation of a cellular proto-oncogene or by expression of a viral oncogene. Examples of such tumor antigens are those expressed on a cancer cells such as melanoma cells, lymphoma cells, Hodgkin's Disease cells, anaplastic large cell cancers, prostate cancer cells, a Burkitt's lymphoma cells, and cervical carcinoma cells, among others.

Examples of specific antibodies or fragments thereof that may be used in the practice of this invention include L8A4, MDX-010 (ipilimumab), HuMax-CD4 (zanolimumab), HuMax-CD20 (ofatumumab), HuMax-EGFR (zalutumumab), MDX-060, MDX-214, CNTO95, MDX-1307, MDX-1106, BMS-66513, IMC-3G3, MDX-1333, Rituxan (Rituximab), Synagis (palivizumab), Herceptin (trastuzumab), Campath-1H (alemtuzumab), Erbitux, Cetuximab, Vectibix (panitumumab), and Avastin.

Another embodiment of this invention utilizes antibodies or fragments thereof that bind an endothelial protein expressed as a result of tumor induced angiogenesis, an example of which is the VEGF receptor.

In some aspects of this invention, the targeting moiety is attached to the protein cage with a linker. Linkers useful in the practice of this invention include homo-bifunctional linker and hetero-bifunctional linkers.

Examples of homo-bifunctional linkers include glutaraldehyde, dimethyl adipimidate (DMA), dimethyl suberimidate (DMS), dimethyl pimelimidate (DMP), N-hydroxysuccinimide (NHS), dithiobis(succinimidylpropionate (DSP), and dithiobis(sulfosuccinimidylpropionate) (DTSSP).

Examples of hetero-bifunctional linker are those with a N-hydroxysuccinimide (NHS) at a first end and a free —SH at a second end, among which, [succinimidyl 3-(2-pyridyldithio)propionate](SPDP) or [succinimidyl trans-4-(maleimidylmethyl)cyclohexane-1-carboxylate](SMCC) are examples.

Generally, linkers can be polymers, peptides, carbohydrates, lipids, or nucleic acids, as well as small molecules. Another feature of some linkers favorable for the practice of this invention is the ability to be cleaved. Thus, linkers cleavable by alkali, acid, reduction, oxidation, protease, electromagnetic radiation, or heat treatment may be used in the practice of this invention.

Examples of proteins suitable to form the protein cages of the present invention include viral and non-viral proteins. In some embodiments, such proteins can be modified. Among the non-viral proteins useful in the practice of this invention are ferritin, apoferritin, a dodecameric cage forming protein, and a heat shock protein (HSP).

In some embodiments of this invention, the protein cages contain a payload comprising at least one therapeutic agent. Examples of therapeutic agents useful in the practice of this invention include anticancer drugs, such as doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine methotrexate, fluorouracils, etoposide, taxol, taxol analogs, tamoxifen, fluorouracil, gemcitabine, and mitomycin. Of particular utility as chemotherapeutic payloads are hypertoxic agents, of which, arsenic oxide, DM1, DM4, Maytansine, dolastatins/auristatins, calichecicins, maytansinoids, CC1065, camptothecin, irinotecan, thiotepa, taxanes, actinomycin, authramycin, azaserines, hemiasterlins, maytansinoids, and esperarnicins are non-limiting examples. In some aspects, the payload includes an inhibitor of the MDR efflux pump in combination with an anticancer agent. In other aspects, the anticancer drug is a radiotherapeutic agent. In further aspects, the payload is siRNA.

Furthermore, in various embodiments, the payload can be crystalline, liquid, or a nanoparticle. Additionally, the payload can be noncovalently associated with the protein cage, by for example, an electrostatic association.

Alternatively, the payload is covalently attached to the protein cage. One form of covalent attachment of the protein cage to the payload is via a linker. Linkers useful for this practice include homo-bifunctional linker and hetero-bifunctional linkers. Examples of homo-bifunctional linkers include glutaraldehyde, dimethyl adipimidate (DMA), dimethyl suberimidate (DMS), dimethyl pimelimidate (DMP), N-hydroxysuccinimide (NHS), dithiobis(succinimidylpropionate (DSP), and dithiobis(sulfosuccinimidylpropionate) (DTSSP). Examples of hetero-bifunctional linker are those with a N-hydroxysuccinimide (NHS) at a first end and a free —SH at a second end, among which, [succinimidyl 3-(2-pyridyldithio)propionate](SPDP) or [succinimidyl trans-4-(maleimidylmethyl)cyclohexane-1-carboxylate](SMCC) are examples.

Generally, linkers can be polymers, peptides, carbohydrates, lipids, or nucleic acids, as well as small molecules. Another feature of some linkers favorable for this practice is the ability to be cleaved. Thus, linkers cleavable by alkali, acid, reduction, oxidation, protease, electromagnetic radiation, or heat treatment may be used for this purpose.

In some embodiments, the protein cage is modulated by a chemical switch. Examples of such chemical switches include those modulated by pH, redox potential, and ionic strength. such chemical switches can be reversible or irreversible.

In further embodiments, a method for treating or inhibiting cancer in a subject is provided by administering to a subject a therapeutically effective amount of a proteinaceous composition comprising a protein cage having a targeting moiety and a payload encapsulated within the protein cage, thereby treating or inhibiting the cancer. Examples of such cancers include Hodgkin's Disease, B-acute lymphoblastic lymphoma, prostate cancer, ovarian cancer, renal cancer, lung cancer, breast cancer, colon cancer, leukemia, multiple myeloma, hepatocarcinoma, Burkitt's lymphoma, and cervical carcinoma. In various aspects, administration is by intravenous infusion or injection.

### BRIEF DESCRIPTION OF THE DRAWINGS

FIG. 1 illustrates (A) three sizes of protein cages, derived from ferritin, HSP, and CCMV coat protein; (B) the reversible gating of cage pores for the loading and unloading of protein cages.

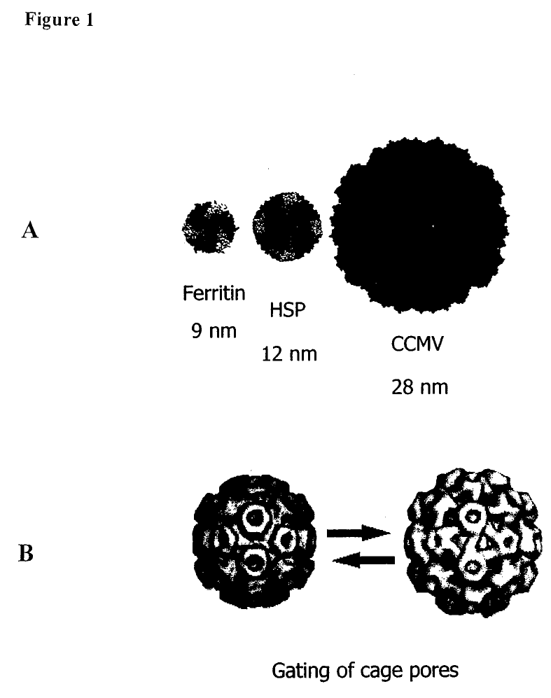

FIG. 2 is a schematic diagram of a protein cage with a targeting moiety.

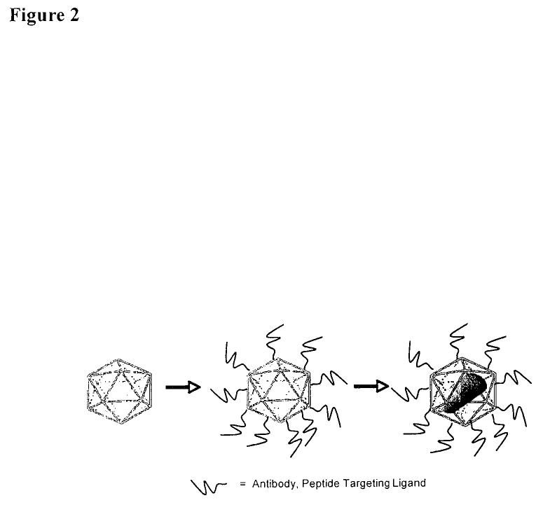

### DETAILED DESCRIPTION OF THE INVENTION

### I. General

A long-standing goal in the pharmacological treatment of disease has been the targeted delivery of therapeutic agents to the site of pathological derangement. Highly targeted drug delivery provides for maximal drug efficacy while avoiding side effects associated with non-specific drug toxicity. The present invention provides a novel method for targeted delivery of a variety of agents by utilizing targeted protein cages containing a payload which can exert a pharmacological or other effect upon reaching a specific destination that is determined by a targeting moiety. Accordingly, in one embodiment, the present invention provides proteinaceous compositions comprising the general structure of a targeting moiety linked to a protein cage and a payload encapsulated within the protein cage. As described in greater detail below, such compositions allow controllable filling and release of the contents (the payload) of a protein cage and highly specific targeting of protein cages to cells and tissues based on the specificity of a targeting moiety. Because of the variety of protein cage materials, payloads that may be encapsulated within the protein cage, and targeting moieties that bind different sites on cells and tissues that may be used, the present invention provides enormous versatility in targeted delivery of a variety of agents to different cells and tissues. This versatility allows the delivery not only of therapeutic agents, but as discussed below, any payload of interest, including for instance, diagnostic and imaging reagents.

### II. Protein Cages

In general, any proteinaceous material that is able to be assembled into a cage-like structure, forming a constrained internal environment, may be used in the practice of this invention. Previous work has utilized several different types of protein “shells” that can be assembled and loaded with different types of materials. For example, protein cages comprising a shell of viral coat protein(s) that encapsulate a non-viral material, as well as protein cages formed from non-viral proteins have been described (see, U.S. Pat. Nos. 6,180,389 and 6,984,386 and U.S. Patent Application 20040028694, incorporated herein in their entity). The compositions of the present invention comprise a proteinaceous shell that self-assembles to form a protein cage (e.g., a structure with an interior cavity which is either naturally accessible to the solvent or can be made to be so by altering solvent concentration, pH, equilibria ratios, as explained below). The protein cages can be loaded with a variety of materials to form a payload as discussed below. Protein cages can have different core sizes, ranging from 1 to 30 nm (e.g., the internal diameter of the shells) with from about 5 to 24 nm being preferred (representing 8.5 to 28 nm outer shell diameters) in some embodiments of the invention.

A. Protein Cages Formed from Viral Coat Proteins

Protein cages can be derived from a variety of viral coat proteins as has been described. In particular, the use of the Cowpea Chlorotic Mottle Virus (CCMV) protein coat exemplifies some of the features of protein cages, which include “controlled gating”, a process which refers to the controlled, reversible or irreversible, formation of an opening large enough to allow atoms and molecules to enter or exit. Accordingly, CCMV has an icosahedral structure approximately 286 angstroms in diameter and is composed of 180 identical protein subunits. (Speir et al., Structure 3: 63 78 (1995)). These subunits are arranged into discrete hexamers and pentamers and self-assemble under the constraints of cubic symmetry to form a roughly spherical structure. Significantly, the native virion undergoes a structural transition in response to changes in pH. (Bancroft et al., Virology 31: 354 379 (1967); Jacrot, Mol. Biol. 95: 433 466 (1975)). As the pH is raised from 5.0 to 7.0 the capsid swells, with an approximately 10 percent increase in diameter. This swelling also induces the formation of openings between the inside and outside of the virion which are approximately 20 angstroms in diameter. Upon lowering the pH, the swollen virion reversibly shrinks, closing off the large openings. Thus, the CCMV virion exemplifies a protein cage in which a gating (i.e., the controlled, reversible opening and closing of the virion) can be readily and reversibly accomplished. The CCMV coat protein can be further modified to yield an extremely stable variant by the introduction of modifications such as inter-subunit disulfide linkages. See U.S. Pat. No. 6,984,386.

While CCMV exemplifies controllable gating and loading of protein cages using pH, controlled gating may be accomplished using other methods, such as, for example, changes in ionic strength, the presence of metal ions and/or chelators, and the like, depending on the composition of the protein cage. See generally Cram et al., “Container Molecules and Their Guests,” Royal Society of Chemistry, Cambridge, England (1994) and Houk et al., Science 273: 627 629 (1996). However, it will be appreciated by the skilled artisan that gating is not necessary to the practice of the invention. As described herein, other means can be used to “trap” agents within protein cages, such as covalent attachment to the interior, non-covalent interactions such as electrostatic interactions, and agent crystallization or precipitation within the core of the protein cage, among other means.

B. Ferritin Protein Cages

Among the non-viral proteins that may be used in the practice of this invention are ferritins and apoferritins, derived from both eukaryotic and prokaryotic species, in particular mammalian and bacterial. Among the ferritins, 12 and 24 subunit ferritins are especially advantageous. Mammalian ferritin is a metalloprotein complex formed from a roughly spherical core containing about 3,000 inorganic atoms such as iron, and a shell of 24 identical subunits each having a molecular weight of about 20 kD. The outer diameter of mammalian ferritin is roughly 12 nm and the core is roughly 8 nm. Ferritin without the iron core molecules is referred to as apoferritin.

C. Protein Cages Formed from Heat Shock Proteins

A number of other known self-assembling “shells”, including various heat shock proteins (HSPs), may be used to form the protein cages of the present invention. Non-limiting examples of such self-assembling shells include the class of 24 subunit heat shock proteins that form an internal core space. Included among the HSPs that find use in the present invention is the small heat shock protein of Methanococcus jannaschii. Members of this family include the dodecameric Dsp heat shock protein of E. coli and the MrgA protein as well as others known in the art.

D. Modification of Protein Cages

As will be appreciated by those in the art, the monomers of the protein cages can be naturally occurring or variant forms, including amino acid substitutions, insertions and deletions (e.g. fragments) that can be made for a variety of reasons as further outlined below. For example, amino acid residues on the outer surface of one or more of the monomers can be altered to facilitate functionalization for attachment to additional moieties (targeting moieties such as antibodies, polymers for delivery, the formation of non-covalent chimeras), to allow for crosslinking (e.g. the incorporation of cysteine residues to form disulfides). Similarly, amino acid residues on the internal surfaces of the shell can be altered to facilitate payload molecule loading, stability, to create functional groups which may be later modified by the chemical attachment of other materials (small molecules, polymers, proteins, etc.).

With respect to some embodiments, in particular, those with dodecameric protein cages, the natural channels to the interior formed by the two-, three-, and four-fold symmetry of the dodecameric proteins may be modified to enable either the introduction and/or extraction, or both, of materials through the opening therein.

It will be appreciated that covalent modifications of protein cages are included within the scope of this invention. One type of covalent modification includes reacting targeted amino acid residues of a cage residue with an organic derivatizing agent that is capable of reacting with selected side chains or the N- or C-terminal residues of a cage polypeptide. Derivatization with bifunctional agents is useful, for instance, for crosslinking the cage to a water-insoluble support matrix or surface for use in the methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl propionate), bifunctional maleimides such as bis-N-maleimido-1,8-octane and agents such as methyl-3-[(p-azidophenyl)dithio]propioimidate. Crosslinking agents find particular use in 2 dimensional array embodiments.

Alternatively, functional groups can be added to the protein cage for subsequent attachment to additional moieties. Preferred functional groups for attachment are amino groups, carboxy groups, oxo groups, and thiol groups. These functional groups can then be attached, either directly or indirectly through the use of a linker. Linkers are well known in the art; for example, homo- or hetero-bifunctional linkers as are well known (see 1994 Pierce Chemical Company catalog, technical section on cross-linkers, pages 155-200, as well as the 2003 catalog, both of which are incorporated herein by reference). Preferred linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred. In some cases, the linkers are cleavable by conditions such as alkali, acid, reduction, oxidation, protease, nuclease or electromagnetic radiation, or heat treatment. See, for example, Flenniken et al., Chem. Comm., 447-449, 2005; Willner et al., Bioconj. Chem. 4:521-7, 1993; U.S. Pat. Nos. 5,767,288 and 4,469,774.

In some embodiments, linkers that influence some property of an attached protein, such as folding, net charge, or hydrophobicity can be used. Other linkers include ones that are cleavable by conditions at the site of action of the payload, such as the pH of a particular cellular compartment, or the presence of a protease. Accordingly, such linkers can be used to attach payload molecules to the interior of protein cages. In some embodiments, the linkers will contain sequences that are cleavable by enzymes or conditions in a cell or tissue targeted by the targeting moiety. Examples of such sequences include those cleaved by cancer specific proteases such as caspases or MMPs. This feature of the linkers will allow for the controlled release of covalently attached payload components at the site of action of a particular reagent, such as for the release of an anticancer agent within a tumor cell.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains (T. E. Creighton, Proteins. Structure and Molecular Properties, W. H. Freeman & Co., San Francisco, pp. 79-86 (1983)), acetylation of the N-terminal amine, and amidation of any C-terminal carboxyl group.

Another type of covalent modification of cages, if appropriate, comprises altering the native glycosylation pattern of the polypeptide. “Altering the native glycosylation pattern” is intended to generally mean deleting one or more carbohydrate moieties found in the native sequence of the cage monomer, and/or adding one or more glycosylation sites that are not present in the native sequence.

Yet another type of covalent modification is to synthesize protein cages with non-natural amino acids that have unique points of conjugation. To effect such modifications, amber codon suppression mutagenesis is used to introduce non-natural amino acids in a site specific manner. The incorporation of non-natural amino acids bearing ketones, azides or alkynes into proteins has been accomplished using this methodology. Such modifications allow further derivatization using hydrozone formation, Staudinger ligation or azide/alkyne cycloaddition reactions, among others. Use of this type of covalent modification allows for specific spatial placement of targeting moieties and controlled stoichiometry. See Chen et al., Current Opinion in Biotechnology 16:35-40, 2005, for review; see, also, Wang L et al., Annu Rev Biophys Biomol Struct. 35:225-49, 2006; Chin et al., J Am Chem Soc 124:9026-9027, 2002.

Addition of glycosylation sites to cage polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Another means of increasing the number of carbohydrate moieties on the polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of cage moieties comprises linking the polypeptide to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337. This finds particular use in increasing the physiological half-life of the composition.

Cage polypeptides of the present invention may also be modified in a way to form chimeric molecules comprising a cage polypeptide fused to another, heterologous polypeptide or amino acid sequence. In one embodiment, such a chimeric molecule comprises a fusion of a cage polypeptide with a tag polypeptide, which provides an epitope to which an anti-tag antibody can selectively bind. The epitope tag is generally placed at the amino- or carboxyl-terminus of the polypeptide. The presence of such epitope-tagged forms of a cage polypeptide can be detected using an antibody against the tag polypeptide. Also, provision of the epitope tag enables the cage polypeptide to be readily purified by affinity purification using an anti-tag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., BioTechnology, 6:1204-1210 (1988)); the KT3 epitope peptide (Martin et al., Science, 255:192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

In a preferred embodiment, the protein cages are derivatized for attachment to a variety of moieties, including but not limited to, dendrimer structures, additional proteins, carbohydrates, lipids, targeting moieties, and the like. In general, one or more of the subunits is modified on an external surface to contain additional moieties.

In a preferred embodiment, the protein cages can be derivatized as outlined herein for attachment to polymers. The character of the polymer will vary, but in certain embodiments, the polymer either contains, or can be modified to contain functional groups for the attachment of the protein cages of the invention. Suitable polymers include, but are not limited to, functionalized dextrans, styrene polymers, polyethylene and derivatives, polyanions including, but not limited to, polymers of heparin, polygalacturonic acid, mucin, nucleic acids and their analogs including those with modified ribose-phosphate backbones, the polypeptides polyglutamate and polyaspartate, as well as carboxylic acid, phosphoric acid, and sulfonic acid derivatives of synthetic polymers; and polycations, including but not limited to, synthetic polycations based on acrylamide and 2-acrylamido-2-methylpropanetrimethylamine, poly(N-ethyl-4-vinylpyridine) or similar quarternized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, spermine, spermidine and polypeptides such as protamine, the histone polypeptides, polylysine, polyarginine and polyomithine; and mixtures and derivatives of these. Particularly preferred polycations are polylysine and spermidine. Both optical isomers of polylysine can be used. The D isomer has the advantage of having long-term resistance to cellular proteases. The L isomer has the advantage of being more rapidly cleared from an animal when administered. As will be appreciated by those in the art, linear and branched polymers may be used.

A preferred polymer is polylysine, as the —NH₂ groups of the lysine side chains at high pH serve as strong nucleophiles for multiple attachment of protein cages. At high pH the lysine monomers can be coupled to the protein cages under conditions that yield on average 5-20% monomer substitution.

The size of the polymer may vary substantially. For example, it is known that some nucleic acid vectors can deliver genes up to 100 kilobases in length, and artificial chromosomes (megabases) have been delivered to yeast. Therefore, there is no general size limit to the polymer. However, a preferred size for the polymer is from about 10 to about 50,000 monomer units, with from about 2000 to about 5000 being particularly preferred, and from about 3 to about 25 being especially preferred.

### III. Targeting Moieties

The present invention provides targeting moieties that direct protein cages to specific molecular and cellular sites. A “targeting moiety” refers to a functional group which serves to target or direct the protein cage complex to a particular location, site, cell type, diseased tissue, or molecular association. In general, the targeting moiety is directed against and binds a target molecule and allows the accumulation of the compositions to a particular location, for instance, to a particular cell type, tissue, or anatomical location within a patient. Thus, for example, antibodies, cell surface receptor ligands and hormones, lipids, sugars and dextrans, alcohols, bile acids, fatty acids, sterols, amino acids, peptides and nucleic acids may all be attached to protein cages to localize or these compositions to a particular site. In one embodiment, the composition is partitioned to the location in a non-1:1 ratio.

A. Antibody Targeting Moieties

An example of an especially advantageous targeting moiety is an antibody. The term “antibody” refers generally to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See, also, Pierce Catalog and Handbook, 1994-1995 (Pierce Chemical Co., Rockford, Ill.). See, also, e.g., Kuby, J., Immunology, 3.sup.rd Ed., W. H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547; Pack and Pluckthun (1992) Biochemistry, 31:1579; Hollinger et al., 1993, supra; Gruber et al. (1994) J. Immunol: 5368; Zhu et al. (1997) Protein Sci 6:781; Hu et al. (1996) Cancer Res. 56:3055; Adams et al. (1993) Cancer Res. 53:4026; and McCartney, et al. (1995) Protein Eng. 8:301.

An antibody immunologically reactive with a particular antigen may be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” refer to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, and the like; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. It will be understood that an epitope can be either a protein, carbohydrate, lipid, nucleic acid, or small molecule entity, although protein epitopes are the most common. In the case of proteins, epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Human antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., BioTechnology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Inter. Rev. Immunol. 13:65-93 (1995).

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, preferably 2, 3, 4, 5, or 6 such sequences. However, it is to be appreciated that some amino acid substitutions within the linker can be made. For example, a valine can be substituted for a glycine.

Methods of making scFv antibodies have been described. See, Huse et al., supra; Ward et al. supra; and Vaughan et al., supra. In brief, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

The antibodies used in the practice of this invention may include bispecific antibodies. Bispecific antibodies are monoclonal, preferably human or humanized, antibodies that have binding specificities for at least two different antigens or that have binding specificities for two epitopes on the same antigen.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J. 10:3655-3659 (1991). Antibody variable domains with the desired binding specificities (antibody-antigen combining sites) can be fused to immunoglobulin constant domain sequences. The fusion preferably is with an immunoglobulin 5 heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology 121:210 (1986).

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

In an advantageous embodiment, the antibody is directed against a cell-surface marker on a cancer cell; that is, the target molecule is a cell surface molecule. As is known in the art, there are a wide variety of antibodies known to be differentially expressed on tumor cells, for example, HER2. Examples of suitable cell surface molecules for antibody binding include CTLA4, CD4, CD20, EGFR, CD30, PSMA, CD89, integrin, mannose receptor/hCGβ, PD1, PDGFR, CD33, CD 5, HER2, CEA, CD13, CD14, CD15, CD33, CD5, CD21, CD19, CD20, CD2, CD3, CD8, CD16, CD 56, and IGFR, among others.

Examples of therapeutic antibodies that may be used in the practice of this invention include L8A4, MDX-010 (ipilimumab), HuMax-CD4 (zanolimumab), HuMax-CD20 (ofatumumab), HuMax-EGFR (zalutumumab), MDX-060, MDX-214, CNTO95, MDX-1307, MDX-1106, BMS-66513, IMC-3G3, MDX-1333, Rituxan (Rituximab), Synagis (palivizumab), Herceptin (trastuzumab), Campath-1H (alemtuzumab), Erbitux, Cetuximab, Vectibix (panitumumab), and Avastin.

In another embodiment, an angiogenic target on, for example, endothelial cells may be recognized by a targeting moiety such as an antibody. Depending on the targeting moiety and the payload carried by a protein cage, the effect of such an interaction could be antiangiogenic. Accordingly, the targeting ligand is specific for tumor induced angiogenic vasculature. In aspects, the targeting ligand can be a tumor vascular homing peptide with tumor cell-penetrating properties. Examples of antiangiogenic targets are the VEGF receptor.

In addition, antibodies against physiologically relevant carbohydrates may be used, including, but not limited to, antibodies against markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In one embodiment, antibodies against virus or bacteria can be used as targeting moieties. As will be appreciated by those in the art, antibodies to any number of viruses (including orthomyxoviruses, (e.g., influenza virus), paramyxoviruses (e.g., respiratory syncytial virus, mumps virus, measles virus), adenoviruses, rhinoviruses, coronaviruses, reoviruses, togaviruses (e.g., rubella virus), parvoviruses, poxviruses (e.g., variola virus, vaccinia virus), enteroviruses (e.g., poliovirus, coxsackievirus), hepatitis viruses (including A, B and C), herpesviruses (e.g., Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g., V. cholerae; Escherichia, e.g. Enterotoxigenic E. coli, Shigella, e.g., S. dysenteriae; Salmonella, e.g., S. typhi; Mycobacterium e.g., M. tuberculosis, M leprae; Clostridium, e.g., C. botulinum, C. tetani, C. dfficile, C. perfringens; Cornyebacterium, e.g., C. diphtheriae; Streptococcus, S. pyogenes, S. pneumoniae; Staphylococcus, e.g., S. aureus; Haemophilus, e.g., H. influenzae; Neisseria, e.g., N. meningitidis, N. gonorrhoeae; Yersinia, e.g., G. lamblia Y. pestis, Pseudomonas, e.g., P. aeruginosa, P. putida; Chlamydia, e.g., C. trachomatis; Bordetella, e.g., B. pertussis; Treponema, e.g., T. palladium; and the like) may be used.

B. Other Targeting Moieties

In some embodiments, in a preferred embodiment, the targeting moiety is all or a portion (e.g., a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF-α and TGF-β, EPO receptor (EPO), TPO receptor (TPO), ciliary neurotrophic factor receptor, prolactin receptor, T-cell receptors, and integrin receptors (e.g., RGD-containing peptides). In particular, hormone ligands are preferred. Hormones include both steroid hormones and proteinaceous hormones, including, but not limited to, epinephrine, thyroxine, oxytocin, insulin, thyroid-stimulating hormone, calcitonin, chorionic gonadotropin, cortictropin, follicle-stimulating hormone, glucagon, leuteinizing hormone, lipotropin, melanocyte-stimutating hormone, norepinephrine, parathyroid hormone, thyroid-stimulating hormone (TSH), vasopressin, enkephalins, seratonin, estradiol, progesterone, testosterone, cortisone, and glucocorticoids and the hormones listed above. Receptor ligands include, but are not limited to, ligands that bind to receptors such as cell surface receptors, which include hormones, lipids, proteins, glycoproteins, signal transducers, growth factors, cytokines, and others.

Furthermore, the targeting moiety can be used to either allow the internalization of protein cage compositions to the cell cytoplasm or localization to a particular cellular compartment, such as the nucleus. For example, the targeting moiety is a nuclear localization signal (NLS). NLSs are generally short, positively charged (basic) domains that serve to direct the moiety to which they are attached to the cell's nucleus. Numerous NLS amino acid sequences have been reported including single basic NLS's such as that of the SV40 (monkey virus) large T Antigen (Pro Lys Lys Lys Arg Lys Val), Kalderon (1984), et al., Cell, 39:499-509; the human retinoic acid receptor-.beta. nuclear localization signal (ARRRRP); NFκB p50 (EEVQRKRQKL; Ghosh et al., Cell 62:1019 (1990); NFκB p65 (EEKRKRTYE; Nolan et al, Cell 64:961 (1991); and others (see, for example, Boulikas, J. Cell. Biochem. 55(1):32-58 (1994), hereby incorporated by reference) and double basic NLS's exemplified by that of the Xenopus (African clawed toad) protein, nucleoplasmin (Ala Val Lys Arg Pro Ala Ala Thr Lys Lys Ala Gly Gln Ala Lys Lys Lys Lys Leu Asp), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey, Ann. Rev. Cell Biol., 2:367-390, 1986; Bonnerot, et al., Proc. Natl. Acad. Sci. USA, 84:6795-6799, 1987; Galileo, et al., Proc. Natl. Acad. Sci. USA, 87:458-462, 1990.

Additional targeting moieties include all or a portion of the HIV-1 Tat protein, and analogs and related proteins, which allows very high uptake into target cells. See for example, Fawell et al., Proc. Natl. Acad. Sci. USA 91:664 (1994); Frankel et al., Cell 55:1189 (1988); Savion et al., J. Biol. Chem. 256:1149 (1981); Derossi et al., J. Biol. Chem. 269:10444 (1994); Baldin et al., EMBO J. 9:1511 (1990); Watson et al., Biochem. Pharmcol. 58:1521 (1999). Targeting moieties for particular organ systems, such as for the hepatobiliary system, may also be used; see U.S. Pat. Nos. 5,573,752 and 5,582,814, both of which are hereby incorporated by reference in their entirety.

In general, targeting moieties can be organic species including biomolecules. In one embodiment, the targeting moiety may be used to either allow the internalization of the protein cage composition to the cell cytoplasm or localize it to a particular cellular compartment, such as the nucleus.

In some embodiments, the targeting moiety can be a peptide. For example, chemotactic peptides have been used to image tissue injury and inflammation, particularly by bacterial infection; see WO 97/14443, hereby expressly incorporated by reference in its entirety. Other peptides useful in the practice of this invention include RGD-containing peptides that bind to integrin receptors and peptides containing the motif NGR, which binds CD13 (a receptor expressed in angiogenic vasculature and in many tumor cell lines), among others. See, e.g., U.S. Patent Application No. 20060275213. Additional peptides useful in the practice of this invention include gastrointestinal tract peptides (GIT) that target and facilitate active uptake across the GI tract.

### IV. Payload Components

In general, any reagent which may be encapsulated within the protein cages of this invention may be used, including for example, small molecule and biomolecule pharmaceutical agents, imaging agents, among others.

A variety of methods may be used to “load” non-native materials into the interior of the protein cages of the present invention. In many embodiments, the protein shells are devoid of their normal cores; e.g. ferritins in the absence of iron (e.g. apoferritins); alternatively, additional loading is done in the presence of some or all of the naturally occurring loading material (if any).

Suitable payload compounds for loading into the protein cages of the present invention include pharmaceutical agents such as chemotherapeutic agents and antimicrobial agents. Particularly suitable chemotherapeutic agents for use in this invention include “hypertoxic” agents, which are agents that are generally considered to non-specifically highly toxic to all cells and require targeting to be therapeutically useful, such as arsenic oxide, DM1, DM4, Maytansine, dolastatins/auristatins, calicheamicin, maytansinoids, CC1065, camptothecin, irinotecan, taxanes, actinomycin, authramycin, azaserines, hemiasterlins, maytansinoids, tubulysin A, and esperamicins, among others. (See, e.g., U.S. Patent Application No. 20040082764.) Other examples of chemotherapeutic agents include: alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, camomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In other embodiments, the pharmaceutical agent is an antiviral or antibacterial drug, including: erythromycin, bacitracin, zinc bacitracin, polymycin, neomycin, chloramphenicol, tetracycline, sulfacetamide, minocycline, clindamycin, doxycycline, undecylenic acid and salts thereof, propionic acid and salts thereof, caprylic acid and salts thereof, ciprofloxacin, cephlasporins, benzoic acid, ciclopiroxolamine, clotrimazole, econazole nitrate, metronidazole, miconazole nitrate, ketacanazole, oxiconazole, tolnaftate, acyclovir, cytarabine, dideoxyadenosine, dideoxycytidine, dideoxyinosine, edoxudine, floxuridine, ganciclovir, idoxuridine, inosine pranobex, MADU, trifluridine, vidarabine and zidovudine, acetylleucine monoethanolamine, amantadine, amidinomycin, cuminaldehyde, thiosemicarbazone, foscamet sodium, interferon-α, interferon-β, interferon-γ, kethoxal, lysozyme, methisazone, moroxydine, podophyllotoxin, ribavirin, rimantadine, stallimycin, statolon, tromantadine and xenazoic acid, among others.

The payload can be also be a radio-sensitizing agent, namely, an agent which sensitizes cells to radiation for use in radiation therapy. Examples of radiosensitizing drugs may include 5-iodo-2′-deoxyuridine and 5-bromo-2′-deoxyuridine.

Other classes of particularly useful payload can include anti-inflammatory agents.

Agents that affect angiogenesis may also be encapsulated within protein cages as payload. Examples of such agents include PAR-1 antagonists, Pazopanib, PTK787, enzastaurin, tyrosine kinase inhibitors, small molecule ATP competitive VEGFR inhibitors, among others. Examples of small molecule VEGFR inhibitors include compounds from distinct chemical classes such as: indolin-2-ones, anilinoquinazolines, anilinophthalazines, isothiazoles, indolo- and indenocarbazoles.

A variety of nucleic acids may be encapsulated into protein cages for targeted delivery. Of particular interest in the practice of this invention are nucleic acids such as antisense nucleic acids, siRNAs, or ribozymes that are able to inhibit the expression of specific genes.

Antisense nucleic acids fall into the categories of enzyme-dependent antisense or steric blocking antisense. Enzyme-dependent antisense includes forms dependent on RNase H activity to degrade a target mRNA, including single-stranded DNA, RNA, and phosphorothioate antisense. Double stranded RNA acts as enzyme-dependent antisense through the RNAi/siRNA pathway, involving target mRNA recognition through sense-antisense strand pairing followed by target mRNA degradation by the RNA-induced silencing complex (RISC). Steric blocking antisense (RNase-H independent antisense) interferes with gene expression or other mRNA-dependent cellular processes by binding to a target sequence of mRNA and getting in the way of other processes. Steric blocking antisense includes 2′-O alkyl (usually in chimeras with RNase-H dependent antisense), peptide nucleic acid (PNA), locked nucleic acid (LNA) and Morpholino antisense.

Small interfering RNA (siRNA), sometimes known as short interfering RNA or silencing RNA, are a class of 20-25 nucleotide-long double-stranded RNA molecules that are involved in the RNA interference (RNAi) pathway by which the siRNA interferes with the expression of a specific gene. Generally, siRNAs are short (usually 21-nt) doubled-stranded RNAs (dsRNAs) with 2-nt 3′ overhangs on either end. (See, generally, Hannon, G. J. et al., Nature, 431, 371-378, 2004.)

Ribozymes that cleave mRNA at site-specific recognition sequences are used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Proteins and peptides may also be encapsulated within the protein cages of the present invention. Examples of useful proteins and peptides include erythropoietins (EPO), talactoferrin, interferons, interleukins, anti-microbial peptides/proteins, insulin, blood-clotting factors, colony-stimulating factors (CSFs), growth hormones, plasminogen activators, reproductive hormones, and therapeutic enzymes, among others.

### V. Formulations and Methods of Treatment

Pharmaceutically acceptable carriers useful for the practice of this invention are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 20^{th} ed., 2003, supra).

Formulations suitable for oral administration can consist of (a) liquid solutions, such as an effective amount of the compound suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the compound with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In some formulations, pharmaceutical preparations deliver one or more of the compounds of the invention, optionally in combination with one or more antiandrogen or chemotherapeutic agents, in a sustained release formulation.

In therapeutic use for the treatment of cancer, the compounds utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of cancer diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

### VI. Examples

The following examples are offered to illustrate, but not to limit the claimed invention.

### 1. Example 1

### Generation of Protein Cages with Targeting Moieties

The small heat shock protein (Mj HSP16.5) (HSP) of Methanococcus jannaschii is subcloned into an appropriate expression vector such as PET-30a(+) (Novagen, Madison, Wis.) for overexpression of the full length protein. In order to facilitate the covalent attachment of reagents of interest to the interior surface of HSP protein cages, glycine 41 is substituted with a cysteine residue by site directed mutagenesis prior to overexpression in E. coli as described (see Flenniken et al., Nano Lett. 3: 1573, 2003). The small HSP is purified from a one liter culture of E. coli as described (see Flenniken et al., Chem. Commun. (Camb.), 447-449, 2005) to yield a preparation of HSP protein cages. The purified small HSP protein cages are conjugated to the monoclonal antibody, Erbitux, using a heterobifunctional cross-linker, sulfosuccinimidyl 4-(N-maleimidomethyl)cyclohexane-1-carboxylate (sulfo-SMCC, Pierce Chemical Company) by first partially reducing the antibody with 10 mM tris(2-carboxyethyl)phosphine (TCEP). Simultaneously, free lysine residues on the small HSP protein cages are reacted with the sulfo-NHS-ester component of the sulfo-SMCC linker added in excess. After incubation for one hour, the HSP protein cage plus linker reaction is subjected to size exclusion chromatography to remove unreacted linker molecules. The reduced Erbitux antibody is then incubated for 3 hours with the SMCC-linkered HSP protein cages, prior to final purification by size exclusion chromatography.

### 2. Example 2

### Loading of Protein Cages with a Therapeutic Agent

The resulting protein cages from Example 1 are loaded with Taxol by utilizing an esterified form of Taxol able to react with cysteine 41 of the modified HSP protein cages. One form of Taxol that may be used for this purpose is the ′2-OH acetylated form of Taxol or the form of Taxol esterified at ′7-OH with N-(4′-fluoresceincarbonyl)-L-alanine group as described (see, Jimenez-Barbero et al., Bioorg. Med. Chem. 6:1857-63, 1998). Thus, HSP protein cages are incubated with an excess of esterified Taxol. After an appropriate reaction time, unreacted Taxol is removed from the reaction by size exclusion chromatography. The extent of covalent attachment of Taxol to the HSP protein cages is verified by liquid chromatography/electrospray mass spectrometry (LC/MS) analysis.

### 3. Example 3

### Generation of Protein Cages with Targeting Moieties Using “Click” Chemistry

The full length heat shock protein (HSP) of Methanococcus jannaschii is overexpressed and purified as discussed in Example 1. All reagent chemicals are used as received. The virus is stored in buffer at a concentration of about 10 mg/mL. HSP concentration is measured by absorbance at 260 nm; HSP at 0.1 mg/mL gives a standard absorbance.

The HSP is functionalized with either an alkyne or an azide conjugate as disclosed in Wang, Q. C. et al., Bioconjugation by copper(I)-catalyzed azide-alkyne [3+2]cycloaddition., J. Am. Chem. Soc., 125, 3192-3193 (2003). A reactive lysine on the exterior of the HSP effectuates the alkyne or azide conjugation using a sulfo-N-hydroxysuccinimide ester of the alkyne or azide conjugate using standard conditions.

After the HSP is functionalized with either an alkyne or azide, an azide or alkyne derivatized Erbitux antibody is added to a solution of the functionalized HSP and CuSO₄ is then added. Following incubation at 4° C. for 16 h to effectuate the “click” chemistry, the mixture is purified by passage through a P-100 size exclusion column (centrifugation at 800 g for 6 min). This filtration is repeated with fresh columns until all the excess reagents is removed (typically twice). The recovery of derivatized HSP antibody is high and concentrations is determined by measuring the absorbance at 260 nm.

The antibody derivativtized HSP can be loaded either before of after antibody derivatization. For example, maleimide functionalized Taxol can be used to react with the interior reactive cysteins of the HSP, or the esterified Taxol can be used as set forth in Example 2.

### 4. Example 4

### CPMV and Ferritin Protein Cages with Targeting Moieties Loaded with Arsenic Trioxide

Cowpea mosaic virus (CPMV) or ferritin is used as the protein component for this Example. CPMV is a structurally rigid assembly of 60 identical copies of a two-protein asymmetric unit around a cavity (see, U.S. Pat. Nos. 6,180,389 and 6,984,386). With respect to ferritin, the 12 or 24 subunit ferritin are equally advantageous.

In this Example, the cage is loaded with the anticancer drug asenic trioxide (As₂O₃) and the payload remains trapped within the protein cage until released inside a tumor. A stable protein cage formulation is made using metal salts such as nickel acetate, cobalt acetate, copper acetate, or zinc acetate and then arsenic trioxide is added. As soon as arsenic trioxide crosses into the cage, it forms an insoluble complex with the metal ions that are already there and remains.

This process produces acetic acid that diffuses out of the cage. As acetic acid leaves the cage, it drives more arsenic trioxide into the cage, further increasing the amount of active drug encapsulated within the cage. The drug may be used to treat acute promyelocytic leukemia.

The cage can also be loaded with a microemulsion of the arsenic trioxide as disclosed in Karasulu et. al., Drug Deliv. 2004 November-December; 11(6):345-50. In the microemulsion formulation, the payload is made up of soybean oil as oil phase, a mixture of Brij 58 and Span 80 as surfactants, absolute ethanol as co-surfactant, and distilled water containing As₂O₃ solution as the aqueous phase. The payload exerts a low cytotoxic effect on normal cells and is effective as an antitumor agent that induces apoptosis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A proteinaceous composition, said proteinaceous composition comprising:
a protein cage having a targeting moiety; and
a payload encapsulated within said protein cage.

2. The composition of claim 1, wherein said targeting moiety is selected from the group consisting of an antibody, a ligand for a receptor, a carbohydrate, a lipid, and a polynucleotide.

3. The composition of claim 2, wherein said targeting moiety comprises an antibody or fragment thereof.

4. The composition of claim 3, wherein said antibody binds a cell surface molecule.

5. The composition of claim 4, wherein said cell surface molecule is selected from the group consisting of growth factor receptors, hormone receptors, lymphocyte surface markers, cell-specific differentiation markers, and cell adhesion molecules.

6. The composition of claim 3, wherein said antibody binds a tumor antigen.

7. The composition of claim 6, wherein said tumor antigen is expressed on a cancer cell selected from the group consisting of a melanoma cell, a lymphoma cell, a Hodgkin's Disease cell, an anaplastic large cell cancer, a prostate cancer cell, a Burkitt's lymphoma cell, and a cervical carcinoma cell.

8. The composition of claim 1, wherein said targeting moiety is attached to the protein cage with a linker.

9. The composition of claim 1, wherein said protein cage comprises a non-viral protein.

10. The composition of claim 9, wherein said non-viral protein comprises a protein selected from the group consisting of ferritin, apoferritin, a dodecameric cage forming protein, and a heat shock protein (HSP).

11. The composition of claim 1, wherein said payload comprises at least one therapeutic agent.

12. The composition of claim 11, wherein said therapeutic agent comprises an anticancer drug.

13. The composition of claim 12, wherein said anticancer drug is a hypertoxic agent.

14. The composition of claim 13, wherein the hypertoxic agent is selected from the group consisting of arsenic oxide, DM1, DM4, Maytansine, dolastatins/auristatins, calicheamicin, maytansinoids, CC1065, camptothecin, irinotecan, thiotepa, taxanes, actinomycin, authramycin, azaserines, hemiasterlins, maytansinoids, and esperamicins.

15. The composition of claim 1, wherein said payload comprises siRNA.

16. A method for treating or inhibiting cancer in a subject, said method comprising:
administering to said subject a therapeutically effective amount of a proteinaceous composition comprising:
a protein cage having a targeting moiety; and
a payload encapsulated within said protein cage,
thereby treating or inhibiting the cancer.

17. The method of claim 16, wherein said cancer is selected from the group consisting of Hodgkin's Disease, B-acute lymphoblastic lymphoma, prostate cancer, ovarian cancer, renal cancer, lung cancer, breast cancer, colon cancer, leukemia, multiple myeloma, hepatocarcinoma, Burkitt's lymphoma, and cervical carcinoma.

18. The method of claim 16, further comprising at least one anticancer agent.

19. The method of claim 18, wherein said at least one anticancer agent is different from the payload.

20. The method of claim 18, wherein said at least one anticancer agent is selected from the group consisting of doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine methotrexate, fluorouracils, etoposide, taxol, taxol analogs, and mitomycin.
